(19) United States
(12) Patent Application Publication
(10) Pub. No.: US 2004/0086965 A1
(43) Pub. Date: May 6, 2004

(54) Casein peptide fragments with growth-influencing activity on cell cultures

(76) Inventors: Juan Claus, Sante Fe (AR); Marcelo Comini, Santa Fe (AR); Georgina Tonarelli, Santa Fe (AR); Juan Carlos Perin, Santa Fe (AR); Jorge Luis Salvetti, Santa Fe (AR); Ronald Frank, Braunschweig (DE)

Correspondence Address: Ronald R Santucci, Frommer Lawrence & Haug, 745 Fifth Avenue, New York, NY, 10151, US

(21) Appl. No.: 10/380,371

(22) PCT Filed: Oct. 9, 2001
(86) PCT No.: PCT/DE01/03849

(30) Foreign Application Priority Data
Oct. 9, 2000 (DE) 100 50 091.9

(51) Int. Cl. C12P 21/02; C12N 5/06; C07K 14/475; C07H 21/04

(52) U.S. Cl. 435/69.1; 435/320.1; 435/325; 530/350; 536/23.5

(57) ABSTRACT

From an enzymatic hydrolysate of bovine casein there was isolated a decapeptide whose sequence could be clearly identified in the primary sequence of the casein. This decapeptide, which was also prepared by synthetic chemistry, influences the growth of the insect cell line IPLB-Sf-21 in a concentration-dependent manner. A tripeptide, which is present twice in the sequence of the decapeptide and the casein, has an even more pronounced action than the decapeptide, that action likewise being concentration-dependent. These peptides can accordingly be used advantageously as additives for serum-free, chemically defined cell culture media.

Increasing use is being made of eukaryotic cell cultures for the biotechnological production of valuable recombinant proteins. Most culture media for such cells have a complex composition and many require the further addition of foetal calf serum (FCS). Because the use of serum in culture media causes problems of many kinds and gives rise to high costs, replacement of the serum component with cheap, defined additives would result in a significant improvement to cell-culture-based biotechnological processes. For the biotechnological production of (recombinant) proteins, there are used, for example, insect cells, BHK (baby hamster kidney) cells, CHO (Chinese hamster ovary) cells, HEK (human epithelial kidney) cells, inter alia.

Protein hydrolysates and natural protein extracts are frequently included as growth-promoting additives in culture media for eukaryotic cells. However, knowledge of their activity is very limited; the active components are not known and their role in influencing growth is unknown. It has already been shown that an enzymatic hydrolysate of bovine casein has a growth-promoting action on cultures of a frequently used insect cell line, IPLB-Sf21 (Spodoptera frugiperda). However, that action is entirely lost in the case of acid hydrolysis.

The problem of the invention is to identify the peptide components of the casein hydrolysate that are responsible for the growth-promoting action of the hydrolysate as a whole and to make available those components in the form of chemically defined additives for culture media for, for example, insect cell cultures.

Consequently, in accordance with patent claim 1, the invention relates to a peptide, having an action of promoting the growth of eukaryotic cell cultures, which is selected from

a) a peptide of the amino acid sequence HQPHQPLPPT,

b) peptides in which one or more amino acid residue(s) in the sequence according to a) is/are replaced by amino acid residues, it being possible, for example, for the degree of homology to be 30%, 40%, 50%, 60%, 70%, 80%, 85%, 90% or 95%,

c) peptides in which one or more amino acid residue(s) in the sequence according to a) or b) is/are chemically modified, it being possible, for example, for the amino acid residue at the N-terminus to have an acyl group such as an acetyl group,

d) peptides in which one or more amino acid residue(s) in the sequence according to a), b) or c) is/are deleted or inserted, it being possible, for example, for 1, 2, 3, 4, 5, 6 or 7 amino acid residues to be deleted,

e) a fusion protein comprising a peptide according to a), b) or d), without the additional amino acid residues impairing the growth-promoting action, or these being readily cleaved off.

Further aspects and or advantageous and/or preferred embodiments of the invention are contained in the subordinate claims or further claims.

The peptide according to the invention in accordance with d) can be the peptide HQP, which can be chemically modified. For example, the amino acid residue at the N-terminus can have an acyl group such as an acetyl group.

The peptides according to the invention can be isolated from natural casein sources (for example, from bovine casein), can be chemically synthesised or can be prepared by genetic engineering.

The invention further relates to a DNA sequence that codes for a fusion protein according to the invention in accordance with e), to a recombinant expression vector that comprises such a DNA sequence, and also to prokaryotic or eukaryotic cells that are transformed or transfected with a DNA sequence according to the invention or an expression vector according to the invention.

In addition, the invention relates to a method of preparing a peptide according to the invention in accordance with a), b) or d) wherein cells according to the invention are grown in a suitable culture medium, the fusion protein is obtained from the cells or the culture medium, and the additional amino acid residues are cleaved off.

The invention moreover relates to a method of growing eukaryotic cell cultures using a peptide according to the invention.

The peptide according to the invention can advantageously be used in a concentration in the range from 0.1 μg per ml to 10 μg per ml of culture medium. Suitable cells are, for example, cells of the insect cell line IPLB-Sf-21.

The invention is described in greater detail hereinbelow with reference to examples, without being limited thereby.

Materials and Methods

Cell Lines and Culture Media

IPLB-Sf-21 cells (ACC1 19, DSMZ in Braunschweig, Germany) were obtained from Dr. Victor Romanowski (Instituto de Bioquímica y Biología Molecular, Facultad de Ciencias Exactas, Universidad Nacional de la Plata, República Argentina) and maintained at 28° C. in TC-100 medium (Gibco/Life Technologies, Grand Island, N.Y., USA) containing 10% foetal calf serum (FCS, SIGMA, St. Louis, Mo., USA).

Casein Hydrolysate

The starting material was purchased from Sigma-Aldrich Co. under the product designation N-Z Case Plus (Order No. N4642). That hydrolysate was first separated into fractions of differing molecular size by means of gel filtration chromatography in a manner known per se, lyophilised, and stored at −20° C. Those fractions were tested with respect to their biological activity. The active fraction was then further fractionated in a manner known per se by reversed-phase high-pressure liquid chromatography (HPLC) using a column measuring 4×250 mm filled with C18-modified silica gel (pore size 300 Å) of 7 μm particle size and using a solvent gradient from water to acetonitrile containing 0.1% trifluoroacetic acid. The column eluate was passed through a UV absorption detector and UV-absorbing compounds were collected in separate fractions. The fractions were concentrated in vacuo; the residue was taken up in 500 μm of water containing 0.1% trifluoroacetic acid and stored at −20° C.

In a manner known per se, a portion of the material from the HPLC fractions was then analysed by mass spectroscopy (matrix-assisted laser desorption ionisation, MALDI).

In the case of the HPLC fractions which had clearly defined peptide masses, a further portion of the material was then characterised in a manner known per se by protein sequencing according to the Edman degradation procedure.

Peptide Synthesis

The peptide fragment that was biologically active and partial segments from the casein hydrolysate were manually prepared by synthetic chemistry using the Fmoc/tBu method (Fields and Noble, Int. J. Peptide Protein Res. 35, 1992, 161-214). As carrier material there was used PEG-polystyrene resin (Rapp Polymere, Tübingen) with a Rink linker. The amino acid derivatives Fmoc-Thr(tBu)-OH, Fmoc-Leu-OH, Fmoc-Pro-OH, Fmoc-His(Trt)-OH, Fmoc-Gln(Trt)-OH (Calbiochem-Novabiochem Corp., California, USA) were linked with benzotriazolyl-1-oxi-tris-pyrrolidinophosphonium hexafluorophosphate (PyBOP) and N-methyl-morpholine (NMM) as catalyst. At the end of the synthesis, the N-terminal amino group of the peptide was blocked using acetic anhydride in DMF as acetamide. The peptides were deprotected using a mixture of trifluoroacetic acid/water/triethylsilane (90/5/5, V/V/V) and cleaved off from the carrier, precipitated in cold diethyl ether, centrifuged off, dissolved in water and freeze-dried. The resulting crude products were then purified in a manner known per se by reversed-phase high-pressure liquid chromatography (HPLC) using a 10×250 mm column filled with C18-modified silica gel of 7 μm particle size and using a solvent gradient from water to acetonitrile containing 0.1% trifluoroacetic acid. Analytical HPLC showed a purity of more than 90%.

The tripeptide was analysed by nuclear magnetic resonance (NMR) spectroscopy.

Determination of the biological activity of the peptides and peptide fractions

IPLB-Sf-21 cells were sown at a starting density of 5×10⁴ cells in 1 ml of culture medium (TC-100+10% FCS) in the individual wells of 24-well culture plates. The plates were incubated at 27° C. for two days in a humidified chamber. During the following two days, the cultures were weaned off the serum additive by replacing the medium several times with pure TC100 medium and were then provided with the peptides or peptide fractions at various concentrations. Control cultures were cultivated in pure TC100 medium.

Four days later, the cells were harvested by being scraped off carefully. The cell count per culture well was determined by counting in a Neubauer chamber with an inverted microscope. The Neubauer chamber had two regions for counting. Each sample was counted three times in each of the two regions (six values per sample).

Results

Peptide Sequences

The main component of the biologically active fraction of the casein was identified as the peptide of the sequence HQPHQPLPPT.

Synthetic Peptides

In accordance with the general method described above, the peptides AC-HQP-OH (tripeptide) and Ac-HQPHQPLPPT-OH (decapeptide) were prepared (Ac denotes acetyl-; amino acids are denoted by the standard single-letter coding).

Influence of the synthetic peptides on cell growth

Table 1 shows the cell yields following treatment with various concentrations of the decapeptide Ac-HQPHQPLPPT-OH in relation to the cell yields in the case of cultures without FCS. The maximum growth-promoting effect was achieved with a peptide concentration of 1 μg/ml. Higher concentrations resulted in growth inhibition.

TABLE 1

Decapeptide concentration (μg/ml)

| | 100 | 10 | 1 | 0.1 | 0.01 |
|---|---|---|---|---|---|
| Rel. yield | 0.866 | 1.175 | 1.272 | 1.182 | 1.051 |
| (p) | (0.056) | (0.058) | (0.118) | (0.332) | (0.157) |

p=standard deviation of error, calculated using the ANOVA (analysis of variance) option of the Microcal Origin program.

The growth-influencing effect of the tripeptide AC-HQP-OH, which is present twice in the casein sequence, is demonstrated in Table 2. The cell yields are again indicated in relation to the cell yield in the case of cultures without peptide and without FCS. In this case, the maximum growth-promoting effect was already achieved with a peptide concentration of 0.1 μg/ml. Very high concentrations (>10 μg/ml) likewise resulted in growth inhibition.

TABLE 2

Tripeptide concentration (μg/ml)

| | 100 | 10 | 1 | 0.1 | 0.01 |
|---|---|---|---|---|---|
| Rel. yield | 0.826 | 1.030 | 1.101 | 1.345 | 1.251 |
| (p) | (0.052) | (0.073) | (0.041) | (0.158) | (0.080) |

1. A peptide, having an action of promoting the growth of eukaryotic cell cultures, which is selected from
a) a peptide of the amino acid sequence HQPHQPLPPT,
b) peptides in which one or more amino acid residue(s) in the sequence according to a) is/are replaced by amino acid residues,
c) peptides in which one or more amino acid residue(s) in the sequence according to a) or b) is/are chemically modified,
d) peptides in which one or more amino acid residue(s) in the sequence according to a), b) or c) is/are deleted or inserted,
e) a fusion protein comprising a peptide according to a), b) or d), without the additional amino acid residues impairing the growth-promoting action, or these being readily cleaved off.

2. A peptide according to claim 1, wherein in the case of the peptide according to b) the degree of homology is 30%, 40%, 50%, 60%, 70%, 80%, 85%, 90% or 95%.

3. A peptide according to claim 1, wherein in the case of the peptide according to d) 1, 2, 3, 4, 5, 6 or 7 amino acid residues are deleted.

4. A peptide according to claim 3, which is the peptide HQP.

5. A peptide according to claim 1, wherein in the peptide according to c) the amino acid residue at the N-terminus has an acyl group.

6. A peptide according to claim 4, wherein in the peptide the amino acid residue at the N-terminus has an acyl group.

7. A peptide according to claim 5 or 6, wherein the acyl group is an acetyl group.

8. A peptide according to one of the preceding claims, which has been isolated from a natural casein source, has been chemically synthesised or has been prepared by genetic engineering.

9. A DNA sequence that codes for a fusion protein according to claim 1 e).

10. A recombinant expression vector that comprises a DNA sequence according to claim 9.

11. A prokaryotic or eukaryotic cell that is transformed or transfected with a DNA sequence according to claim 9 or an expression vector according to claim 10.

12. A method of preparing a peptide according to claim 1 a), b) or d), wherein a cell according to claim 11 is grown in a suitable culture medium, the fusion protein is obtained from the cells or the culture medium and the additional amino acid residues are cleaved off.

13. A method of growing eukaryotic cell cultures using a peptide according to one of claims 1 to 8.

14. A method according to claim 13, wherein a peptide according to one of claims 1 to 8 is used in a concentration in the range from 0.1 μg per ml to 10 μg per ml of culture medium.

15. A method according to claim 13 or 14, wherein the cells are the insect cell line IPLB-Sf-21.


SEQUENCE LISTING (1 sequence)

SEQ ID NO: 1 - protein, 10 aa, Artificial Sequence
  Description of Artificial Sequence Synthetic peptide
HQPHQPLPPT